(19) United States
(12) Patent Application Publication
(10) Pub. No.: US 2023/0313105 A1
(43) Pub. Date: Oct. 5, 2023

(54) Additive Manufacturing of a Packed Bed Bioreactor

(71) Applicant: The Government of the United States of America, as represented by the Secretary of the Navy, Arlington, VA (US)

(72) Inventors: Matthew Yates, Chantilly, VA (US); Sarah Glaven, Washington, DC (US)

(21) Appl. No.: 17/712,989

(22) Filed: Apr. 4, 2022

(51) Int. Cl. C12M 1/12; C12M 1/00; B33Y 10/00; B33Y 80/00

(52) U.S. Cl. CPC C12M 25/18; C12M 23/40; C12M 27/22; C12M 23/06; B33Y 10/00; B33Y 80/00

(57) ABSTRACT

A packed bed bioreactor is fabricated as a monolith entirely using additive manufacturing techniques, also known as 3D printing. Construction of a bioreactor in this manner enables control over the reactor dimensions and properties (such as void volume) as well as the dimensions, shape, and pattern of the media bed. Together these attributes give the end-user control over the size, shape, material, and flow characteristics of the bioreactor.

### FEDERALLY-SPONSORED RESEARCH AND DEVELOPMENT

The United States Government has ownership rights in this invention. Licensing inquiries may be directed to Office of Technology Transfer, US Naval Research Laboratory, Code 1004, Washington, DC 20375, USA; +1.202.767.7230; techtran@nrl.navy.mil, referencing NC 210504.

### CROSS-REFERENCE TO RELATED APPLICATIONS

None.

### BACKGROUND

Microorganisms can be harnessed in different biotechnologies to perform useful functions, such as the removal of waste from water or the synthesis of various products. The advantage of using biology for product synthesis is that microorganisms can catalyze reactions with high selectivity and specificity. Further, the development and maturation of synthetic biology tools that enable control over microbial processes give a greater level of control and larger suite of products that can be made using biosynthesis. As such, using microorganisms as a production platform to generate different products has received increased attention in recent years.

Efficient bioprocesses are enabled by optimized reactor design, construction, and operational parameters. Traditional bioreactors comprise large stainless steel vessels to enable sterilization and minimize corrosion during biosynthesis. Fabrication of these reactors is relatively time consuming and energy intensive, resulting in reactors that cannot be easily adapted to new processes or technologies. These large-scale reactors predominantly utilize microorganisms in the planktonic state to maximize mass transfer of products and reactants within the reactor. Utilizing planktonic cells also often necessitates larger bioreactors to maintain desired production rates. These considerations are necessary for industrial-scale biosynthesis, but do not enable adaptable, reconfigurable systems that can be utilized in decentralized/small-scale applications, due to size, weight, space, and cost requirements.

Additive manufacturing, commonly referred to as 3D printing, is a powerful tool that allows precise control over the design and configuration of the fabricated material. To date, a variety of materials have been used for additive manufacturing, including various plastics and metals, and a number of different techniques have been developed for additive manufacturing, each with distinct advantages and disadvantages. Additive manufacturing has been explored as a method to make reactors for a variety of different applications.

A need exists for new applications for additive manufacturing.

### BRIEF SUMMARY

In a first embodiment, a monolithic packed bed bioreactor includes an inlet port; an outlet port; a media bed comprising a plurality of packing elements enclosed within the reactor shell and between the ports; a dispersion plate between the inlet port and the media bed and configured to distribute fluid flowing from the inlet port across the media bed; and a reactor shell enclosing the media bed, wherein the ports, reactor shell, dispersion plate, and packing elements form a single monolithic structure; and wherein the media bed is retained in the reactor shell solely by being connected thereto.

In another embodiment, a method of making a packed bed bioreactor includes providing a set of computer-readable instructions to a 3D printer describing a reactor according to the first embodiment, and causing the printer to make the reactor.

In a further embodiment, the packing elements have one or more shapes occurring in a continuously repeating array and the shapes of those packing elements in the array that are in contact with an inner wall of the reactor shell are truncated where they merge into the wall.

In a yet another embodiment, the packing bed possesses a void fraction of at least 75% and a specific surface area of at least 650 m²/m³.

### BRIEF DESCRIPTION OF THE DRAWINGS

FIGS. 1A and 1B provide cutaway views of an exemplary packed bed bioreactor.

FIG. 2 is a photograph showing Marinobacter atlanticus biofilm growing on a 3D printed Nylon reactor half (upper portion of image). In this case, only half of the bioreactor was printed to facilitate viewing of the internal media bed. Further, the cells were modified to express a red fluorescent protein to facilitate visualization of the cells on the Nylon. An unused half reactor is shown in white in the lower portion of the image.

### DETAILED DESCRIPTION

### Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

Overview

Described here is a packed bed bioreactor fabricated entirely using additive manufacturing techniques. Construction of a bioreactor in this manner enables control over the reactor dimensions and properties as well as the dimensions, shape, and pattern of the media bed. Together these attributes give the end-user control over the size, shape, material, and flow characteristics of the bioreactor as well as placement of ports for desired sensors.

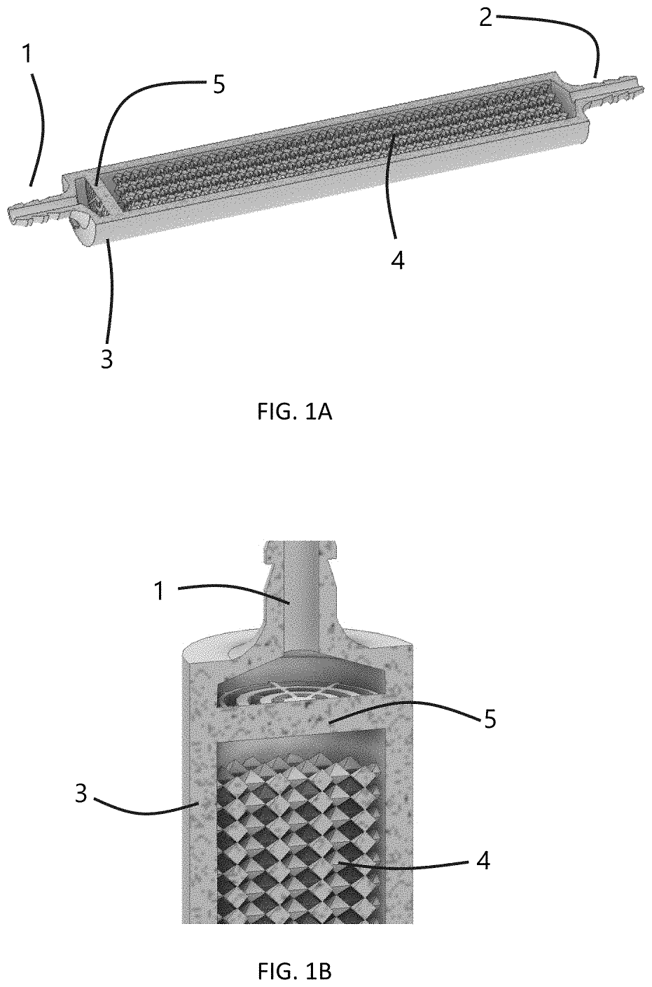

FIGS. 1A and 1B depict an exemplary monolithic packed bed bioreactor in cutaway view. Opposing inlet port 1 and outlet port 2 are attached to the reactor shell 3 which in turn contains a media bed 4 comprising a plurality of packing elements enclosed within the reactor shell. Fluid flows from the inlet port and reaches the dispersion plate 5 which spans the diameter of the reactor and is also embedded in the reactor walls. The dispersion plate 5 operates to help ensure even distribution of fluid flow into and across the media bed 4. The packing elements in the media bed 4 can be colonized by microbes.

The packing elements are interconnected by the 3D printing process to form a monolithic media bed. The shapes and dimensions of the packing elements can be controllably varied in order to achieve the desired porosity (also called void fraction) and specific surface area throughout a wide range, limited primarily by the additive manufacturing process used. For instance, while the below example featured a specific surface area of approximately −650 m²/m³, it is expected that values of 750 m²/m³, 1000 m²/m³, or even greater could be obtainable, while maintaining porosity sufficient for a desired fluid flow rate in a colonized reactor.

In one embodiment, the media bed comprises packing elements of having one or more shapes in a continuously repeating array. In a further embodiment, seen in FIG. 1B, the shapes of those packing elements in the array that are in contact with the inner reactor wall are truncated where they merge into the wall.

By virtue of the manufacturing process, the media bed is fixed in the reactor shell and retained therein without requiring other means of containment, such as a filter or sieve, that would be needed in other reactor designs. The material used for the packing should be compatible with the growth of cells. In various embodiments, the packing material should also resist chemicals employed in potential downstream processing steps, such as product extraction.

The design of the media bed should provide sufficient rigidity and stability to withstand handling of the reactor as well as expected flow through the reactor. In the case of a media bed with packing elements in a continuously repeating array, overlapping the elements can increase the rigidity and stability of the media bed.

### Example

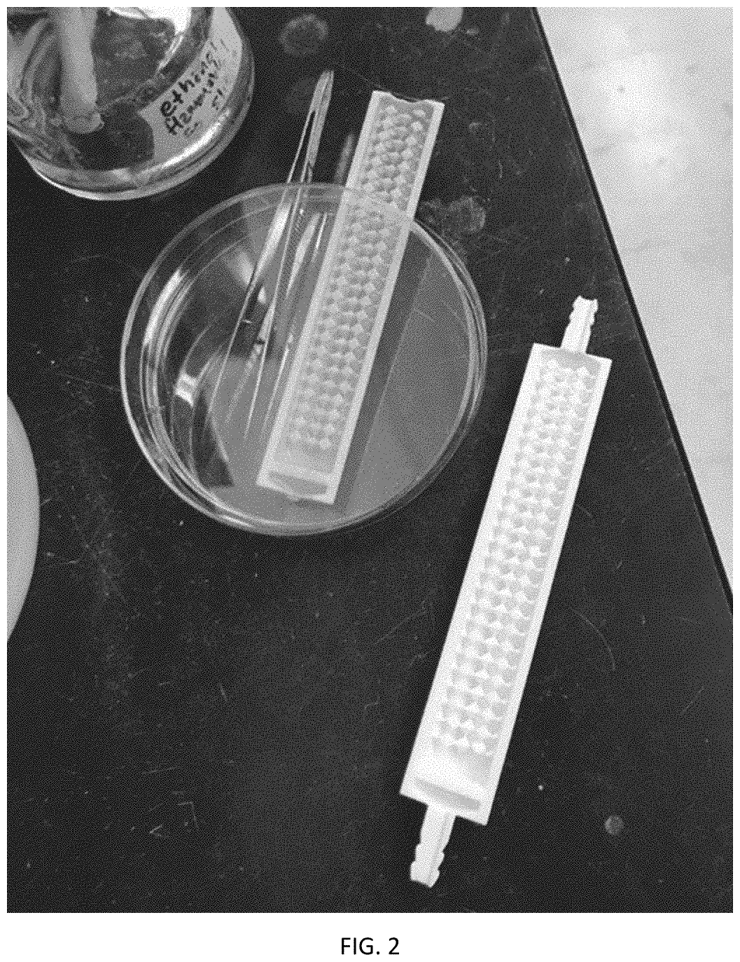

A packed bed bioreactor in the format shown in FIGS. 1A and 1B was made from a polyamide, namely Nylon 6,6, using a selective laser sintering (SLS) 3D printer. The reactor shell was approximately 120 mm in length and the packing elements were regular octrahedra about 2 mm in size that were partially overlapping, providing a media bed with a specific surface area of approximately −650 m²/m³ and a porosity of 83%. As seen in FIG. 2, Marinobacter atlanticus, a marine organism isolated from the Biocathode MCL community (see U.S. Patent Application Pub. No. 2021/0253990) successfully formed a biofilm on the reactor material (compare upper colonized cutaway reactor to lower un-colonized cutaway reactor).

The polyamide material was exposed to seawater for a period of at least five days without observed degradation.

### Further Embodiments

The packing elements can have practically any shape, such as polygonal, spherical, octahedral, etc. Aspects of the shapes can be further varied, for example with octahedra the height can be independently varied from the center of the shape to the point of the pyramidal components. The width can also be varied independently from the center to the edge of the square on the midplane of the shape. The shapes of the packing elements can overlap to form connections therebetween. The packing elements can be smooth or can include pores or other texture in order to increase surface area for cell growth.

In one embodiment, interconnections between the individual packing elements are substantially smaller than the largest dimensions of the individual elements. For example, packing elements having a largest dimension of 2 mm might be interconnected by bridges with a diameter of 0.1 mm. In another embodiment, the packing elements are filaments of substantially uniform or varying diameter that optionally intersect one another. The filaments can have cross sections that are substantially circular, oval, polyhedral, and so forth.

The ports can be made to include desired fittings which can be the same or different from one another. For example, the ports can be configured to include barbed fittings as in the example, or quick connect, cam-lock, or other types of fittings. In various aspects, the ports can be configured to receive fittings that are separate from the 3D printed monolith. Additional ports can be included in order to introduce reactants (such as oxygen or air), to remove samples, and/or to allow for the placement of sensors in the reactor.

The reactor shell can be cylindrical as in the example, or can have other shapes. Reactor dimensions that can be varied based on the needs of the application, whether that is height, surface area, volume, diameter, etc. within the limits of the 3D printer being used.

A variety of 3D printing (additive manufacturing) techniques can be used to make the bioreactor, including selective laser sintering (SLS), fused deposition modeling (FDM), stereolithography (SLA) or digital light processing (DLP).

### Advantages

The porosity, also termed void volume, of the media bed can be easily and tightly controlled. The 3D printing process provides provide an extremely high degree of control over this value as compared to a traditional packed bed reactor, where it tends to have an undesirably large distribution. Furthermore, having completely controllable channel sizes enables a level of porosity sufficient to prevent blockage of the channels due to microbial growth, which otherwise can build up back pressure in the reactor and requires backwashing and re-equilibration to alleviate the blocking in the reactor. Computer-aided design and 3D printing enable one to desirably maximize surface area of the column while maintaining sufficient porosity.

It is expected that additively manufactured bioreactors will enable biosynthesis for decentralized and distributed applications, such as synthesis of materials in strategic locations for military applications and humanitarian response efforts. The ability to synthesize materials in the field will alleviate logistical burdens of supply and resupply to mission in the field. Utilizing an additively manufactured system will also enable different reactors to be brought to the field in the form of computer-aided design (CAD) files. The CAD file containing reactor specifications that best suit the local conditions can then be selected and printed on-site, instead of trying to adapt or retrofit an existing reactor in the field. In addition, the desired scale of production can be controlled by implementing a modular design to maintain a desired production rate. As 3D printers become more common as part of the available set of tools in the field, the materials and printers will already be on hand. This design will leverage these technologies to tailor fabrication of a biosynthesis reactor to field conditions and available materials.

### CONCLUDING REMARKS

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

What is claimed is:

1. A monolithic packed bed bioreactor comprising:
an inlet port;
an outlet port;
a media bed comprising a plurality of packing elements enclosed within the reactor shell and between the ports;
a dispersion plate between the inlet port and the media bed and configured to distribute fluid flowing from the inlet port across the media bed; and
a reactor shell enclosing the media bed,
wherein the ports, reactor shell, dispersion plate, and packing elements form a single monolithic structure; and
wherein the media bed is retained in the reactor shell solely by being connected thereto.

2. The bioreactor of claim 1, wherein said packing elements have one or more shapes occurring in a continuously repeating array and wherein the shapes of those packing elements in the array that are in contact with an inner wall of the reactor shell are truncated where they merge into the wall.

3. The bioreactor of claim 1, wherein the media bed possesses a void fraction of at least 75% and a specific surface area of at least 650 m²/m³.

4. The bioreactor of claim 1, wherein said monolithic structure comprises polyamide.

5. A method of making a packed bed bioreactor, the method comprising:
providing a set of computer-readable instructions to a 3D printer, where the instructions encode the production of a packed bed bioreactor comprising an inlet port, an outlet port, a media bed comprising a plurality of packing elements enclosed within the reactor shell and between the ports, a dispersion plate between the inlet port and the media bed and configured to distribute fluid flowing from the inlet port across the media bed, and a reactor shell enclosing the media bed, wherein the ports, reactor shell, dispersion plate, and packing elements form a single monolithic structure; and wherein the media bed is retained in the reactor shell solely by being connected thereto; and
causing the printer to print the packed bed bioreactor.

6. The method of claim 5, wherein said packing elements have one or more shapes occurring in a continuously repeating array and wherein the shapes of those packing elements in the array that are in contact with an inner wall of the reactor shell are truncated where they merge into the wall.

7. The method of claim 5, wherein the media bed possesses a void fraction of at least 75% and a specific surface area of at least 650 m²/m³.

8. The method of claim 5, wherein said packed bed bioreactor is formed of polyamide.
